(19) United States
(12) Patent Application Publication
(10) Pub. No.: US 2004/0147452 A1
(43) Pub. Date: Jul. 29, 2004

(54) Non-amphoteric glutathione derivative compositions for tropical application

(76) Inventors: Ruey J. Yu, Ambler, PA (US); Eugene J. Van Scott, Abington, PA (US)

Correspondence Address: AKIN GUMP STRAUSS HAUER & FELD L.L.P., ONE COMMERCE SQUARE, 2005 MARKET STREET, SUITE 2200, PHILADELPHIA, PA, 19103-7013, US

(21) Appl. No.: 10/626,158

(22) Filed: Jul. 24, 2003

Related U.S. Application Data
(60) Provisional application No. 60/400,252, filed on Jul. 31, 2002.

(51) Int. Cl. A61K 38/05

(52) U.S. Cl. 514/18

(57) ABSTRACT

Topical compositions and methods including non-amphoteric derivatives of glutathione, for example, N-acyl-glutathiones, N-acyl-glutathione amides, and N-acyl-glutathione esters are disclosed for use in the treatment and prevention of cosmetic conditions and dermatological disorders, are disclosed. The non-amphoteric glutathione derivatives may have the structure of (I):

R¹—COCHNH(R²)CH₂CH₂CONHCH(CH₂SR³)CONHCH₂CO—R¹ (I)

wherein R¹ is independently selected from —OH, —NH₂, —NHNH₂, an alkoxyl group, an aralkoxyl group, and an aryloxyl group and R² and R³ are each independently selected from a hydrogen atom or an acyl group, but if at least one R¹ is —OH, —NH₂, or —NHNH₂, then R² is an acyl group.

### CROSS-REFERENCE TO RELATED APPLICATIONS

This application claims priority under 35 U.S.C. § 119(e) to U.S. provisional patent application serial No. 60/400,252, filed Jul. 31, 2002, the contents of which are incorporated herein by reference.

### BACKGROUND OF THE INVENTION

Glutathione (GSH) in reduced form is a tripeptide (γ-Glu-Cys-Gly) consisting of three amino acid units, namely glutamic acid, cysteine, and glycine (C₁₀ H₁₇ N₃ O₆ S, molecular weight 307, mp 195°). The oxidized glutathione (GSSG) is a dimer with two molecules linked by a dithio bond. In contrast to most peptide linkages that are formed between an alpha amino group and an adjacent carboxyl group, the linkage between the glutamic acid residue and the cysteine residue in glutathione is from the gamma carboxyl group, not the alpha carboxyl group, of glutamic acid. This unique structure makes glutathione a physiologically significant tripeptide.

Native glutathione is present in cells and tissues of the animal body and performs a variety of physiological functions including cellular detoxification, transport and metabolic processes. For example, glutathione helps destroy toxic peroxides and free radicals, reduces methemoglobin to hemoglobin in red blood cells, is involved in leukotriene biosynthesis, and maintains the sulfhydryl groups of intracellular proteins. Thus, glutathione is required for maintenance of healthy cells and tissues of the animal and the human body.

Because glutathione has one free amino group (at the alpha position in glutamic acid) and two free carboxyl groups (one at the alpha position in glutamic acid and the other at the alpha position in glycine), this tripeptide exists in amphoteric form in an aqueous solution (a positively charged amino group and two negatively charged carboxyl groups). By “amphoteric” it is meant a substance that contains both acidic and alkaline radicals or groups in the same molecule and that is ionized with negative and positive charges when it is in solution. Glutathione monoesters and glutathione monoamides are also amphoteric substances because they each have one free amino group and one free carboxyl group.

In normal, healthy, human skin, the stratum corneum consists of approximately fourteen to thirty layers of corneocytes including the inner level (stratum compactum) and the outer level (stratum dysjunctum). The keratin-enriched corneocytes in the stratum corneum are embedded in a lipid matrix and are very resistant to penetration by ionic or amphoteric substances, or by molecules with molecular weights greater than approximately 800 to 1,000. Glutathione, glutathione monoesters, and glutathione monoamides are ionic amphoteric substances and therefore cannot readily penetrate the intact human skin. Thus, these tripeptides are therapeutically ineffective for topical treatment of cosmetic conditions and dermatological disorders.

Nonetheless, the prior art describes attempts to use glutathione or glutathione monoesters in various topical treatments. For example, a process for treating the scalp and skin, characterized by an excessive secretion of sebum has been disclosed to improve the condition by topical application of a composition comprising S-substituted glutathione in the prior art. A cosmetic or dermatological composition containing glutathione monoester is disclosed for topical treatment of cutaneous ageing. Additionally, in the prior art, a process for topical treatment of cutaneous ageing using a composition containing glutathione mono-alkyl ester is disclosed. However, the conventional prior art treatments do not involve topical use of compositions containing non-amphoteric glutathione derivatives in topical treatments that are able to penetrate intact skin.

### SUMMARY OF THE INVENTION

It has been discovered that compositions containing non-amphoteric glutathione derivatives are useful in the treatment and/or prevention of cosmetic conditions and dermatological disorders of the skin, hair, nails, and mucosal surfaces when applied topically. The invention described herein provides compositions for topical administration that includes (a) a non-amphoteric glutathione derivative and (b) a topically acceptable vehicle. The non-amphoteric glutathione derivative may be an N-acyl-glutathione, an N-acyl-glutathione amide, and/or an N-acyl-glutathione ester.

In another embodiment, the invention provides a method for the treatment and/or prevention of cosmetic conditions and/or dermatological disorders that entails topical administration of the non-amphoteric glutathione derivative-containing compositions to an affected area of a patient.

### DETAILED DESCRIPTION OF THE INVENTION

It has been discovered that chemical modification of amphoteric glutathione to substantially eliminate its amphotericity results in tripeptide derivatives that are therapeutically effective for prevention and treatment of various cosmetic conditions and dermatological indications, including cosmetic and clinical signs of aging, oxidative stress, and other related damages to human skin, when administered topically. More specifically, the invention relates to compositions that include non-amphoteric glutathione derivatives (one or more) and a topically acceptable vehicle; these compositions are for use in the treatment of varying cosmetic conditions and/or dermatological disorders. Also included within the scope of the invention are methods of using the compositions in the treatment of the disorders.

The non-amphoteric glutathione derivatives that may be used in the methods or compositions of the invention include any that are modified so that the native amphoteric character of the glutathione is substantially reduced or eliminated, such that the resultant non-amphoteric tripeptide is capable of penetrating intact human skin for the prevention and/or treatment of various cosmetic conditions and/or dermatological disorders. The selected non-amphoteric glutathione derivative may be an oxidized derivative or a reduced derivative; however, reduced derivatives are preferred. Without wishing to be bound by theory, it is hypothesized that the compositions and methods of the invention are effective in the treatment of various cosmetic conditions and dermatological disorders because the non-amphoteric glutathione derivatives are either active themselves or are converted to active glutathione by enzymatic hydrolysis or deacetylation, upon penetration of the skin.

Non-amphoteric glutathione derivatives for use in the invention may include those having the structure represented by the formula (I):

R¹—COCHNH(R²)CH₂CH₂CONHCH(CH₂SR³)CONHCH₂CO—R¹ (I)

The groups represented by RI may be independently selected from —OH, —NH₂, —NHNH₂, an alkoxyl group, an aralkoxyl group, and an aryloxyl group. If R¹ is an alkoxyl group, aralkoxyl group, and/or an aryloxyl group, such group(s) preferably has one to nine carbon atoms, more preferably one to three carbon atoms such as a methyl group, an ethyl group, a propyl group, or an isopropyl group.

The groups represented by R² and R³ in formula (I) may be independently selected from a hydrogen atom or an acyl group. If R² or R³ is an acyl group, the group preferably has two to nine carbon atoms, most preferably two to three carbon atoms, such as an acetyl group or a propanoyl group. However, in the non-amphoteric glutathione derivatives of the invention, when at least one R¹ is —OH, —NH₂, or —NHNH₂, R² is not a hydrogen atom, but is an acyl group.

Non-amphoteric glutathione derivatives for use in the composition and methods of the invention may be linear or branched, although linear is preferred. They may be substituted or unsubstituted. By substituted it is meant, for example, that any hydrogen atom attached to a carbon atom or a nitrogen atom may be substituted by another atom or group, including, for example, a halogen (such as a fluorine atom, an iodine atom, a chlorine atom, a bromine atom) or an alkoxy group having one to nine carbon atoms, preferably one to three carbon atoms.

It is preferred that the non-amphoteric glutathione derivative selected for use in the compositions and/or methods of the invention is an N-acyl-glutathione, an N-acyl-glutathione amide, and/or an N-acyl-glutathione ester. Representative non-amphoteric glutathione derivatives for use in the compositions and methods of the invention may include, but are not limited to, N-acetyl-glutathione monoamide, N-acetyl-glutathione diamide, N-acetyl-glutathione monomethyl ester, N-acetyl-glutathione dimethyl ester, N-acetyl-glutathione monoethyl ester, N-acetyl-glutathione diethyl ester, N-acetyl-glutathione monopropyl ester, N-acetyl-glutathione dipropyl ester, N-acetyl-glutathione monoisopropyl ester, N-acetyl-glutathione diisopropyl ester, N-propanoyl-glutathione, N-propanoyl-glutathione monoamide, N-propanoyl-glutathione diamide, N-propanoyl-glutathione monomethyl ester, N-propanoyl-glutathione dimethyl ester, N-propanoyl-glutathione monoethyl ester, N-propanoyl-glutathione diethyl ester, N-propanoyl-glutathione monopropyl ester, N-propanoyl-glutathione dipropyl ester, N-propanoyl-glutathione monoisopropyl ester, and N-propanoyl-glutathione diisopropyl ester.

Other suitable non-amphoteric glutathione derivatives include, for example, N,S-diacetyl-glutathione; N,S-diacetyl-glutathione monoamide; N,S-diacetyl-glutathione diamide; N,S-diacetyl-glutathione monomethyl ester; N,S-diacetyl-glutathione dimethyl ester; N,S-diacetyl-glutathione monoethyl ester; N,S-diacetyl-glutathione diethyl ester; N,S-diacetyl-glutathione monopropyl ester; N,S-diacetyl-glutathione dipropyl ester; N,S-diacetyl-glutathione monoisopropyl ester; N,S-diacetyl-glutathione diisopropyl ester; N,S-dipropanoyl-glutathione; N,S-dipropanoyl-glutathione monoamide; N,S-dipropanoyl-glutathione diamide; N,S-dipropanoyl-glutathione monomethyl ester; N,S-dipropanoyl-glutathione dimethyl ester; N,S-dipropanoyl-glutathione monoethyl ester; N,S-dipropanoyl-glutathione diethyl ester; N,S-dipropanoyl-glutathione monopropyl ester; N,S-dipropanoyl-glutathione dipropyl ester; N,S-dipropanoyl-glutathione monoisopropyl ester; N,S-dipropanoyl-glutathione diisopropyl ester; glutathione diamide; glutathione dimethyl ester; glutathione diethyl ester; glutathione dipropyl ester; glutathione diisopropyl ester; N-acetyl-S-propanoyl-glutathione; and N-propanoyl-S-acetyl-glutathione.

Non-amphoteric glutathione derivatives of the invention may be present as isomers of the structures recited above, as well as in the form of free acids, salts, partial salts, amides or esters. If the selected non-amphoteric glutathione derivative is a glutathione diester, such as glutathione dimethyl ester, it may be present in a salt form, for example, as a hydrochloride, a sulfate, or a nitrate, to increase the stability of the compound in an aqueous environment. However, in such cases it is preferred that the glutathione diester salt-containing composition also incorporates one or more amino acids, preferably a basic amino acid, to enhance the bioavailability of the glutathione diester. Exemplary amino acids suitable for this use include arginine, lysine, histidine, tryptophan, ornithine, derivatives of the same, and short polypeptides of the same (for example, one to ten residue polypeptides).

In general, the non-amphoteric glutathione derivatives of the invention are present in the compositions in an amount sufficient to enable delivery of a therapeutically effective amount of the non-amphoteric glutathione derivatives to the area affected by the cosmetic condition and/or dermatological condition, by administration of a reasonable dosage quantity via a reasonable dosage regime. Accordingly, the concentration or amount of non-amphoteric glutathione derivative present in the composition will necessarily vary, depending on the chemical and physical properties of the topical vehicle used, as well as those of the specific non-amphoteric glutathione derivative selected.

For example, the composition may contain about 0.01% to about 99.9% of the non-amphoteric glutathione derivative(s), by weight of the total composition. It is preferred that the composition contain about 0.1% to about 30% by weight of the non-amphoteric glutathione derivative(s), more preferred that it contain about 1% to about 20% of the non-amphoteric glutathione derivative(s) by weight of the total composition, and most preferred that it contain about 2% to about 10% by weight of the total composition.

The compositions of the invention include a topical vehicle. Any vehicle acceptable for topical use, known or to be developed in the art, may be used. Preferred topical vehicles include, water, ethanol, propylene glycol, butylene glycol, diisopropyl adipate, nonoxynols, oleyl lactate, triethyl citrate and permethyls.

In addition, the compositions of the invention may contain other cosmetic, pharmaceutical and/or topical agents to enhance, improve or otherwise add to the efficacy or stability of the composition, to increase or add to the overall therapeutic effect of the compositions or the agents, or to otherwise increase the comfort or treatment regime compliance of the patient. The nature, amount and types of agent(s) selected will be variable depending on the chemical and physical properties, and the therapeutic effects desired in the end composition as well as the site to which the composition is to be administered.

Any agents known or to be developed that are suitable for use in a topically administered preparation may be incorporated into the compositions of the invention. For example, suitable cosmetic, pharmaceutical or other topical agents may include aclovate, acyclovir, acetylsalicylic acid, adapalene, albuterol, aluminum acetate, aluminum chloride, aluminum hydroxide, aluminum chlorohydroxide, amantadine, aminacrine, aminobenzoic acid (PABA), aminocaproic acid, aminosalicylic acid, amitriptyline, anthralin, ascorbic acid, ascoryl palimate, atropine, azelaic acid, bacitracin, bemegride, beclomethasone dipropionate, benzophenone, benzoyl peroxide, betamethasone dipropionate, betamethasone valerate, brompheniramine, bupivacaine, butoconazole, calcipotriene, camphor, capsaicin, carbamide peroxide, chitosan, chlorhexidine, chloroxylenol, chlorpheniramine, ciclopirox, clemastine, clindamycin, clioquinol, clobetasol propionate, clotrimazole, coal tar, cromolyn, crotamiton, cycloserine, dehydroepiandrosterone, desoximetasone, dexamethasone, diphenhydramine, doxypin, doxylamine, dyclonine, econazole, erythromycin, estradiol, ethinyl estradiol, fluocinonide, fluocinolone acetonide, 5-fluorouracil, griseofulvin, guaifenesin, haloprogin, hexylresorcinol, homosalate, hydrocortisone, hydrocortisone 21-acetate, hydrocortisone 17-valerate, hydrocortisone 17-butyrate, hydrogen peroxide, hydroquinone, hydroquinone monoether, hydroxyzine, ibuprofen, ichthammol, imiquimod, indomethacin, ketoconazole, ketoprofen, kojic acid, lidocaine, meclizine, meclocycline, menthol, mepivacaine, methyl nicotinate, methyl salicylate, metronidazole, miconazole, minocycline, minoxidil, monobenzone, mupirocin, naftifine, naproxen, neomycin, nystatin, octyl methoxycinnamate, octyl salicylate, oxybenzone, oxiconazole, oxymetazoline, padimate O, permethrin, pheniramine, phenol, phenylephrine, phenylpropanolamine, piperonyl butoxide, podophyllin, podofilox, povidone iodine, pramoxine, prilocaine, procaine, promethazine propionate, propranolol, pseudoephedrine, pyrethrin, pyrilamine, resorcinol, retinal, 13-cis retinoic acid, retinoic acid, retinol, retinyl acetate, retinyl palmitate, salicylamide, salicylic acid, selenium sulfide, shale tar, sulconazole, sulfur, sulfadiazine, tazarotene, terbinafine, terconazole, tetracaine, tetracycline, tetrahydrozoline, thymol, tioconazole, tolnaftate, triamcinolone diacetate, triamcinolone acetonide, triamcinolone hexacetonide, triclosan, triprolidine, undecylenic acid, urea, vitamin E acetate, wood tar, zinc pyrithione, lactobionic acid, maltobionic acid, glycolic acid, mandelic acid, lactic acid, tropic acid, methyllactic acid, tartaric acid, citric acid, malic acid, glucuronic acid, glucuronolactone, ribonic acid, ribonolactone, gluconic acid, gluconolactone, galactonic acid, galactonolactone, glucarolactone, galactarolactone, N-acetyl-cysteine, N-acetyl-proline, N-acetyl-prolinamide, N-acetyl-lysine, N-acetyl-glutamine, N-acetyl-ornithine and N-acetyl-glucosamine.

Other topical agents that may be combined with non-amphoteric glutathione derivative composition include hydroxycarboxylic acids, polyhydroxyacids, polyhydroxylactones, O-acetyl-hydroxycarboxylic acids, oligosaccharide aldonic acids, N-acylamino sugars, N-acylamino acids, hydroxyacids, ketoacids and related compounds, phenyl alpha acyloxyalkanoic acids and derivatives, N-acetyl-aldosamines, N-acetylamino acids and related N-acetyl compounds, local analgesics and anesthetics, antibacterials, antiyeast agents, antifungal agents, antiviral agents, antihistamine agents, antipruritic agents, antiemetics, antimotion sickness agents, anti-inflammatory agents, vitamins, corticosteroids, hormones, gum disease or oral care agents, triclosan, sodium fluoride, zinc chloride, zinc citrate, zinc sulfate, chlorhexidine and chlorhexidine digluconate.

The compositions of the invention may be prepared as various formulations suitable for topical administration, such as, liquid or semi-liquid preparations, emulsions, liniments, lotions, oil-in-water or water-in-oil emulsions, creams, ointments, pastes, solutions or suspensions, or may be incorporated into microparticles that are suspended in a vehicle. Other formulations into which the compositions may be prepared include aqueous or non-aqueous solutions, gels, shampoos, sprays, sticks, powders, masques, mouth rinses or washes, vaginal gels or preparations, or other forms acceptable for use on skin, nail, hair, oral mucosa, vaginal mucosa, nasal mucosa, or anal mucosa.

In general, regardless of the chosen formulation, a composition may be prepared by dissolving one or more non-amphoteric glutathione derivatives of the instant invention in a solution prepared from water, ethanol, propylene glycol, butylene glycol, and/or other topically acceptable vehicle. To prepare a topical composition in lotion, cream or ointment form, the selected glutathione derivative is first dissolved in a solvent, such as water, ethanol, propylene glycol, and/or another vehicle, and the solution thus obtained is mixed with a desired base or pharmaceutically acceptable vehicle to make a lotion, a cream or an ointment. Concentrations of the glutathione derivative can be the same as described above.

A topical composition of the instant invention may also be formulated in a gel or shampoo form. A typical gel composition is formulated by the addition of a gelling agent such as chitosan, methyl cellulose, ethyl cellulose, polyvinyl alcohol, polyquaterniums, hydroxyethylcellulose, hydroxypropylcellulose, hydroxypropylmethylcellulose, carbomer, or ammoniated glycyrrhizinate to a solution comprising the glutathione derivative. In formulating a gel, the preferred concentration of the gelling agent may range from about 0.1% to about 4% by weight of the total composition.

In the preparation of shampoo, the glutathione derivative is first dissolved in water or propylene glycol, and the solution thus obtained is mixed with a shampoo base. Concentrations of the glutathione derivative used in gel or shampoo form may be the same as described above.

The compositions of the invention including the non-amphoteric glutathione derivatives can be used in the treatment and or prevention of numerous cosmetic conditions and/or dermatological disorders, including psoriasis, ichthyosis, eczema, other inflammatory diseases of the skin or mucosa, and disturbed keratinization of the skin. Additionally, because of the antioxidant and other physiological functions carried out by non-amphoteric glutathione derivatives, the compositions of the invention are topically effective for prevention and treatment of erythema, edema, exfoliation and numerous other skin changes or damage caused by ultraviolet radiation.

The compositions of the invention containing non-amphoteric glutathione derivatives are beneficial for use as preventive measures or for their topical effects on skin, hair, nail, gums; and the mucosa of the oral, vaginal and anal cavities, to alleviate or improve various cosmetic conditions, for general care purposes, for healing of skin wounds, and for other dermatological disorders, including dry skin, acne, and signs of aging, changes or damage to skin, nails and hair associated with intrinsic aging and/or extrinsic aging, as well as changes or damage caused by extrinsic factors such as sunlight, air pollution, wind, cold, heat, dampness, chemicals, smoke, cigarette smoking, laser treatment, and radiation, including electromagnetic radiation and ionizing radiation.

General cosmetic conditions and dermatological indications that may be treated by the compositions and the methods of the invention may include inflammation or disturbed keratinization of the skin, defective synthesis of dermal components, dryness or looseness of skin, nail and hair, xerosis, palmar and plantar hyperkeratosis, uneven and rough surface of skin, nail and hair, dandruff, Darier's disease, lichen simplex chronicus, keratoses, acne, pseudofolliculitis barbae, dermatoses, eczema, psoriasis, pruritus, warts, herpes and other viral infections, age spots, lentigines, melasmas, blemished skin, mottled skin, hyperkeratosis, hyperpigmented or darker skin, abnormal or diminished syntheses of collagen, glycosaminoglycans, proteoglycans and elastin as well as diminished levels of such components in the dermis, stretch marks, skin lines, fine lines, wrinkles, thinning of skin, nail plate and hair, skin thickening due to elastosis of photoaging, loss or reduction of skin, nail and hair resiliency, elasticity and recoilability, lack of skin, nail and hair lubricants and luster, dull and older-looking skin, nail and hair, fragility and splitting of nail and hair, or used as in skin lightening.

Specific skin changes associated with aging which may be treated with the compositions and methods of the invention. For example, progressive thinning of skin, fragile skin, deepening of skin lines and fine lines, wrinkles including fine and coarse wrinkles, lusterless skin surface, coarse and uneven skin, loss of skin elasticity and recoilability, blemished and leathery skin, loss of skin lubricating substances, increased numbers of blotches and mottles, nodules, pre-cancerous lesions, pigmented spots and mottled skin, changes in qualities and quantities of collagen and elastic fibers, solar elastosis, decrease in collagen fibers, diminution in the number and diameter of elastic fibers in the papillary dermis, atrophy of the dermis, stretch marks, reduction in subcutaneous adipose tissue and deposition of abnormal elastic materials in the upper dermis, yellowing skin, telangiectatic skin, and older-looking skin may be treated using the compositions and methods of the invention.

Non-amphoteric glutathione derivative compositions and methods of the invention are also useful in promoting wound healing and in the general care of skin, hair, and nails; oral, vaginal and anal mucosa; and gum diseases. General care includes prevention, maintenance and treatment of skin, nails and hair; oral, vaginal and anal mucosa; against erythema, inflammation, itching, irritation caused by internal or external factors, including sunlight, radiations, ionizing radiations, air pollution, wind, cold, dampness, heat, chemicals, smoke, and cigarette smoking.

The non-amphoteric glutathione derivative compositions and methods of the invention are useful to provide wound healing of skin, irritated or inflamed mucosa or skin; for skin lightening; for cleansing and conditioning of skin, hair and nail; for protection from extrinsic factors; for mouthwashes; for use as antioxidant agent, toner, cleanser, moisturizer, emollient, protectant, foundation makeup, beauty masks, face powders, rouge, concealer make-up (“cover-up”), lipsticks, eye makeup, dentifrices, suntan or sunscreen preparations, soap preparations; as a skin refinisher, to improve skin pores, flakiness and to reduce redness; to make skin soft, smooth, fresh, balanced, firm, visibly clear, even-toned and brighter.

When the compositions according to the present invention are used for general care, as well as treatment and prevention of diseases and conditions of the oral, vaginal, and anal mucosa, other suitable agents may be incorporated to provide synergistic or additional therapeutic effects.

These topical agents include hydroxyacids, ketoacids and related compounds, phenyl alpha acyloxyalkanoic acids and derivatives, N-acetyl-aldosamines, N-acetylamino acids and related N-acetyl compounds, local analgesics and anesthetics, antibacterials, antiyeast agents, antifungal agents, antiviral agents, antihistamine agents, antipruritic agents, antiemetics, antimotion sickness agents, anti-inflammatory agents, vitamins, corticosteroids, hormones, and gum disease or oral care agents. Some examples are triclosan, sodium fluoride, zinc chloride, zinc citrate, zinc sulfate, chlorhexidine and chlorhexidine digluconate.

When the compositions according to the present invention are used for treating skin wounds in aiding the healing of skin cuts, tears, lacerations, burns, punctures, and other wounds, other topical agents may be added. These agents include hydroxyacids, polyhydroxy acids, polyhydroxy lactones, ketoacids and related compounds, phenyl alpha acyloxyalkanoic acids and derivatives, N-acetyl-aldosamines, N-acetylamino acids and related N-acetyl compounds, analgesics and anesthetics, wound cleansers, antibacterials, antiyeast agents, antifungal agents, antiviral agents, anti-inflammatory agents, vitamins, burn relief agents, and corticosteroids.

To prepare a combination composition for synergistic or additive effects, a cosmetic, pharmaceutical or other topical agent is incorporated into any one of the above compositions by dissolving or mixing the agent into the formulation. Other forms of compositions for delivery of glutathione derivative of the instant invention are readily blended, prepared or formulated by those skilled in the art.

Furthermore, a method for treating cosmetic conditions and dermatological disorders comprising topically applying a therapeutically effective amount of a composition comprising at least one compound selected from the group consisting of non-amphoteric glutathione derivatives, their free acids, esters, amides, salt or partial salt form in a topically acceptable vehicle is provided.

In one embodiment of the invention, the method comprises topically applying a therapeutically effective amount of a composition comprising at least one compound selected from the group consisting of non-amphoteric glutathione derivatives, their free acid, ester, amide, salt or partial salt form, and at least one cosmetic, pharmaceutical, or other topical agent in a topically acceptable vehicle.

### EXAMPLES

In Use Examples 2 to 4, skin thicknesses were measured by micrometer calipers using the following method: The skin was grasped with a 2×6 cm metal hinge, the internal faces of which were coated with emery cloth to prevent slippage, and manually squeezed to threshold patient discomfort. The combined thickness of two whole-skin layers including thickness of the two hinge leaves was measured with micrometer calipers. The thickness of the two hinge leaves was subtracted to determine the actual thickness of two whole-skin layers. Triplicate measurements on treated sites were done and an average number was used for calculation of the skin thickness.

### Comparative Example 1

The antioxidant capacity of a specific non-amphoteric glutathione derivative in comparison to ascorbic acid and glutathione (both known antioxidants) is evaluated by a qualitative assessment of each test material's ability to prevent or retard air oxidation of an anthralin (0.1%) oil-in-water cream.

An oil-in-water cream containing 0.1% anthralin is prepared by conventional methods. As is known in the art, anthralin is highly susceptible to oxidation when exposed to air. Unoxidized anthralin and/or compositions containing unoxidized anthralin appear yellow in color. As oxidation occurs, the color of the anthralin composition changes from yellow to gray, then to brown.

To carry out the evaluation, four aliquots (numbered Tubes 1, 2, 3 and 4) of 100 gms of the 0.1% anthralin cream are measured out. The test materials are added to each of the aliquots, as shown in Table I:

TABLE I

| Tube | Test Material |
|---|---|
| 1 | none |
| 2 | ascorbic acid |
| 3 | glutathione (amphoteric) |
| 4 | glutathione diamide (non- |
| | amphoteric) |

In the case of Tubes 2-4, the final concentration of test material is 1% by weight of the total composition.

The four tubes are maintained, capless, in the environment of an open room at approximately 21° C. Every six hours a visual observation is made.

After twenty-four hours, it is observed that the material of Tube 1 appears gray; the material in the remaining Tubes 2-4 remains yellow. After one hundred forty-four hours of exposure, the material in each of Tubes 2-4 begins to change from yellow to gray. The results of this experiment demonstrate that the glutathione derivative used in the compositions and methods of the invention possesses similar antioxidant properties as ascorbic acid and amphoteric glutathione.

### Preparation Example 1

A solution containing a non-amphoteric glutathione derivative was prepared. Glutathione dimethyl ester hydrochloride 10 g was dissolved in 88.5 ml of a solution prepared from (i) water 40 parts, (ii) ethanol 40 parts, and (iii) propylene glycol 20 parts by volume. L-Arginine 1.5 g was added.

The resulting formulation had a pH of 5.6 and contained 10% non-amphoteric glutathione dimethyl ester in a bioavailable form.

### Preparation Example 2

A solution containing a non-amphoteric glutathione derivative was prepared. Glutathione dimethyl ester hydrochloride 10 g was dissolved in water 18 ml, and L-arginine 2 g was added. The solution was mixed uniformly with 70 g cream base. The resultant cream had a pH of about 5.9 and contained 10% non-amphoteric glutathione dimethyl ester in a bioavailable form.

### Preparation Example 3

A solution containing the non-amphoteric glutathione derivative of the invention was prepared. Glutathione diamide 5 g was dissolved in water 20 ml and propylene glycol 20 ml. The resulting solution was mixed uniformly with 55 g cream base. The glutathione diamide-containing cream had a pH of 5.7 and contained 5% non-amphoteric glutathione diamide in a bioavailable form.

### Preparation Example 4

A solution containing a non-amphoteric glutathione derivative was prepared. Glutathione diethyl ester 5 g was dissolved in warm propylene glycol 20 ml. The resulting solution was mixed uniformly with a cream base or hydrophilic ointment 75 g. The cream had a pH of 3.4 and contained 5% non-amphoteric glutathione diethyl ester in a bioavailable form.

### Preparation Example 5

A solution containing a non-amphoteric glutathione derivative was prepared. N-Acetyl-glutathione diethyl ester 5 g was dissolved in ethanol 10 ml and propylene glycol 20 ml. The solution thus obtained was mixed uniformly with a cream base or hydrophilic ointment 65 g. The cream thus formulated had pH 3.5 and contained 5% non-amphoteric N-acetyl-glutathione diethyl ester in a bioavailable form.

### Preparation Example 6

A solution containing a non-amphoteric glutathione derivative was prepared. N-Acetyl-glutathione 5 g was dissolved in warm water 20 ml and the solution thus obtained was mixed uniformly with a cream base or hydrophilic ointment 75 g. The cream thus formulated had pH 4.4 and contained 5% non-amphoteric N-acetyl-glutathione in a bioavailable form.

### Use Example 1

A male subject, age 70, suffering chronic nummular eczema on his right lower leg over a period of several years, topically applied twice daily the non-amphoteric glutathione dimethyl ester (10%)-containing cream of Preparation Example 2 on the eczematous lesions for two days. At the end of two days, the areas of the eczematous lesions were reduced by 75%. This demonstrates that non-amphoteric glutathione dimethyl ester is therapeutically effective for topical treatment of eczema.

### Use Example 2

Glutathione dimethyl ester hydrochloride 5 g and L-arginine 1 g were dissolved in 94 ml solution prepared from ethanol 40 parts, water 40 parts and propylene glycol 20 parts. The solution thus prepared contained 5% non-amphoteric glutathione derivative. For comparative study, glutathione 5 g and L-arginine 1 g were dissolved in 94 ml solution prepared from water 70 parts and propylene glycol 30 parts. The solution thus prepared contained 5% amphoteric glutathione.

A female subject, age 66, applied topically twice daily the above 5% amphoteric glutathione solution to her left forearm and 5% non-amphoteric glutathione derivative to her right forearm for four weeks. After four weeks her left forearm was still loose, relatively thin and wrinkled when lifted. In contrast, her right forearm was more firm, smooth, plump and minimally wrinkled when lifted. While there was no change in skin thickness of her left forearm, her right forearm had increased 10% in skin thickness as measured by the micrometer calipers. This result indicated that non-amphoteric glutathione dimethyl ester would be therapeutically effective for topical treatment of wrinkles, photoaging and other changes of skin, nail or hair associated with aging.

### Use Example 3

A solution containing a non-amphoteric glutathione derivative was prepared. Glutathione dimethyl ester hydrochloride 5 g and L-arginine 1 g were dissolved in 94 ml solution prepared from ethanol 40 parts, water 40 parts and propylene glycol 20 parts. The solution thus prepared contained 5% non-amphoteric glutathione derivative.

A female subject, age 62, applied topically twice daily the above 5% non-amphoteric glutathione derivative to her right forearm for eight weeks. After eight weeks her untreated left forearm was still loose, relatively thin and wrinkled when lifted. In contrast, her right forearm was more firm, smooth, plump and minimally wrinkled when lifted. While there was no change in skin thickness of her left forearm, her right forearm had increased 44% in skin thickness as measured by the micrometer calipers. This result indicated that non-amphoteric glutathione dimethyl ester would be therapeutically effective for topical treatment of wrinkles, photoaging and other changes of skin, nail or hair associated with aging.

### Use Example 4

A female subject, age 50, applied topically twice daily the above 5% non-amphoteric glutathione derivative to her left forearm for eight weeks. After eight weeks her untreated right forearm was still loose, relatively thin and wrinkled when lifted. In contrast, her left forearm was more firm, smooth, plump and minimally wrinkled when lifted. While there was no change in skin thickness of her right forearm, her left forearm had increased 20% in skin thickness as measured by the micrometer calipers. This result indicated that non-amphoteric glutathione dimethyl ester would be therapeutically effective for topical treatment of wrinkles, photoaging and other changes of skin, nail or hair associated with aging.

### Use Example 5

A solution containing a non-amphoteric glutathione derivative was prepared. N-Acetyl-glutathione diethyl ester 3 g was dissolved in warm propylene glycol 17 ml, and the solution was mixed with 80 g oil-in-water cream base or hydrophilic ointment. The cream contained 3% non-amphoteric N-acetyl-glutathione diethyl ester.

A male subject, age 31, who had X-linked ichthyosis with severe dry skin participated in this study. Test spots were I cm square sites on extensor surface of forearm, a grid pattern formed by Hayes Test Chambers on Hayes adhesive strips. Each test chamber contained a square piece of filter paper which was fully saturated with the non-amphoteric glutathione derivative cream. Test chambers were impressed on the skin to leave outlines which were marked with SANFORD® SHARPIE® permanent marker. Sites were re-marked at each successive application of the test cream. Chambers were removed twice weekly, and replaced with a new adhesive strip of chambers with filter paper saturated with the test cream. The test was carried out for two weeks. After one week of topical application, the rough and scaly skin disappeared and clinical evaluation was judged to be 90% improvement. After two weeks of topical application, the severe dry skin became smooth and normal-looking. This result indicated that N-acetyl-glutathione diethyl ester was therapeutically effective for topical treatment of ichthyosis and severe dry skin conditions.

It will be appreciated by those skilled in the art that changes could be made to the embodiments described above without departing from the broad inventive concept thereof. It is

understood, therefore, that this invention is not limited to the particular embodiments disclosed, but it is intended to cover modifications within the spirit and scope of the present invention as defined by the appended claims.

We claim:

1. A composition for topical administration comprising:
(a) a non-amphoteric glutathione derivative and
(b) a topically acceptable vehicle.

2. The composition of claim 1, wherein the non-amphopteric glutathione derivative is selected from the group consisting of an N-acyl-glutathione, an N-acyl-glutathione amide, and an N-acyl-glutathione ester.

3. The composition of claim 1, wherein the non-amphopteric glutathione derivative is selected from the group consisting of N-acetyl-glutathione; N-acetyl-glutathione monoamide; N-acetyl-glutathione diamide; N-acetyl-glutathione monomethyl ester; N-acetyl-glutathione dimethyl ester; N-acetyl-glutathione monoethyl ester; N-acetyl-glutathione diethyl ester; N-acetyl-glutathione monopropyl ester; N-acetyl-glutathione dipropyl ester; N-acetyl-glutathione monoisopropyl ester; and N-acetyl-glutathione diisopropyl ester.

4. The composition of claim 1, wherein the non-amphopteric glutathione derivative is selected from the group consisting of N-propanoyl-glutathione; N-propanoyl-glutathione monoamide; N-propanoyl-glutathione diamide; N-propanoyl-glutathione monomethyl ester; N-propanoyl-glutathione dimethyl ester; N-propanoyl-glutathione monoethyl ester; N-propanoyl-glutathione diethyl ester; N-propanoyl-glutathione monopropyl ester; N-propanoyl-glutathione dipropyl ester; N-propanoyl-glutathione monoisopropyl ester; and N-propanoyl-glutathione diisopropyl ester.

5. The composition of claim 1, wherein the non-amphopteric glutathione derivative is selected from the group consisting of N,S-diacetyl-glutathione; N,S-diacetyl-glutathione monoamide; N,S-diacetyl-glutathione diamide; N,S-diacetyl-glutathione monomethyl ester; N,S-diacetyl-glutathione dimethyl ester; N,S-diacetyl-glutathione monoethyl ester; N,S-diacetyl-glutathione diethyl ester; N,S-diacetyl-glutathione monopropyl ester; N,S-diacetyl-glutathione dipropyl ester; N,S-diacetyl-glutathione monoisopropyl ester; and N,S-diacetyl-glutathione diisopropyl ester.

6. The composition of claim 1, wherein the non-amphopteric glutathione derivative is selected from the group consisting of N,S-dipropanoyl-glutathione; N,S-dipropanoyl-glutathione monoamide; N,S-dipropanoyl-glutathione diamide; N,S-dipropanoyl-glutathione monomethyl ester; N,S-dipropanoyl-glutathione dimethyl ester; N,S-dipropanoyl-glutathione monoethyl ester; N,S-dipropanoyl-glutathione diethyl ester; N,S-dipropanoyl-glutathione monopropyl ester; N,S-dipropanoyl-glutathione dipropyl ester; N,S-dipropanoyl-glutathione monoisopropyl ester; N,S-dipropanoyl-glutathione diisopropyl ester; glutathione diamide; glutathione dimethyl ester; glutathione diethyl ester; glutathione dipropyl ester; glutathione diisopropyl ester; N-acetyl-S-propanoyl-glutathione; and N-propanoyl-S-acetyl-glutathione.

7. The composition of claim 1, wherein the composition further comprises one or more cosmetic, pharmaceutical or other topical agent.

8. The composition of claim 7, wherein the cosmetic, pharmaceutical or other topical agent is selected from the group consisting of agents that improve or eradicate age spots, keratoses and wrinkles; local analgesics and anesthetics; antiacne agents; antibacterials; antiyeast agents; antifungal agents; antiviral agents; antidandruff agents; antidermatitis agents; antihistamine agents; antipruritic agents; antiemetics; antimotionsickness agents; antiinflammatory agents; antihyperkeratolytic agents; antiperspirants; antipsoriatic agents; antiseborrheic agents; hair conditioners and hair treatment agents; antiaging and antiwrinkle agents; sunblock and sunscreen agents; skin lightening agents; depigmenting agents; vitamins; corticosteroids; tanning agents; humectants; hormones; retinoids; gum disease or oral care agents; topical cardiovascular agents; corn, callus and wart removing agents; and dipilating agents.

9. The composition of claim 7, wherein the cosmetic, pharmaceutical or other topical agent is selected from the group consisting of aclovate, acyclovir, acetylsalicylic acid, adapalene, albuterol, aluminum acetate, aluminum chloride, aluminum hydroxide, aluminum chlorohydroxide, amantadine, aminacrine, aminobenzoic acid (PABA), aminocaproic acid, aminosalicylic acid, amitriptyline, anthralin, ascorbic acid, ascoryl palimate, atropine, azelaic acid, bacitracin, bemegride, beclomethasone dipropionate, benzophenone, benzoyl peroxide, betamethasone dipropionate, betamethasone valerate, brompheniramine, bupivacaine, butoconazole, calcipotriene, camphor, capsaicin, carbamide peroxide, chitosan, chlorhexidine, chloroxylenol, chlorpheniramine, ciclopirox, clemastine, clindamycin, clioquinol, clobetasol propionate, clotrimazole, coal tar, cromolyn, crotamiton, cycloserine, dehydroepiandrosterone, desoximetasone, dexamethasone, diphenhydramine, doxypin, doxylamine, dyclonine, econazole, erythromycin, estradiol, ethinyl estradiol, fluocinonide, fluocinolone acetonide, 5-fluorouracil, griseofulvin, guaifenesin, haloprogin, hexylresorcinol, homosalate, hydrocortisone, hydrocortisone 21-acetate, hydrocortisone 17-valerate, hydrocortisone 17-butyrate, hydrogen peroxide, hydroquinone, hydroquinone monoether, hydroxyzine, ibuprofen, ichthammol, imiquimod, indomethacin, ketoconazole, ketoprofen, kojic acid, lidocaine, meclizine, meclocycline, menthol, mepivacaine, methyl nicotinate, methyl salicylate, metronidazole, miconazole, minocycline, minoxidil, monobenzone, mupirocin, naftifine, naproxen, neomycin, nystatin, octyl methoxycinnamate, octyl salicylate, oxybenzone, oxiconazole, oxymetazoline, padimate O, permethrin, pheniramine, phenol, phenylephrine, phenylpropanolamine, piperonyl butoxide, podophyllin, podofilox, povidone iodine, pramoxine, prilocaine, procaine, promethazine propionate, propranolol, pseudoephedrine, pyrethrin, pyrilamine, resorcinol, retinal, 13-cis retinoic acid, retinoic acid, retinol, retinyl acetate, retinyl palmitate, salicylamide, salicylic acid, selenium sulfide, shale tar, sulconazole, sulfur, sulfadiazine, tazarotene, terbinafine, terconazole, tetracaine, tetracycline, tetrahydrozoline, thymol, tioconazole, tolnaftate, triamcinolone diacetate, triamcinolone acetonide, triamcinolone hexacetonide, triclosan, triprolidine, undecylenic acid, urea, vitamin E acetate, wood tar, zinc pyrithione, lactobionic acid, maltobionic acid, glycolic acid, mandelic acid, lactic acid, tropic acid, methyllactic acid, tartaric acid, citric acid, malic acid, glucuronic acid, glucuronolactone, ribonic acid, ribonolactone, gluconic acid, gluconolactone, galactonic acid, galactonolactone, glucarolactone, galactarolactone, N-acetyl-cysteine, N-acetyl-proline, N-acetyl-prolinamide, N-acetyl-lysine, N-acetyl-glutamine, N-acetyl-ornithine and N-acetyl-glucosamine.

10. A composition for topical administration comprising:
(a) a non-amphoteric glutathione derivative represented by the formula (I):
R¹—COCHNH(R²)CH₂CH₂CONHCH(CH₂SR³)CONHCH₂CO—R¹ (I)
wherein R¹ is independently selected from —OH, —NH₂, —NHNH₂, an alkoxyl group, an aralkoxyl group, and an aryloxyl group and R² and R³ are each independently selected from a hydrogen atom or an acyl group, but if at least one R¹ is —OH, —NH₂, or —NHNH₂, then R² is an acyl group; and
(b) a topically acceptable vehicle. x.x.x

11. The composition of claim 10, wherein R¹ is independently selected from an alkoxyl group having one to nine carbon atoms, an aralkoxyl group having one to nine carbon atoms, and an aryloxyl group having one to nine carbon atoms.

12. The composition of claim 10, wherein R² or R³ is independently an acyl group having two to nine carbon atoms.

13. The composition of claim 10, wherein the composition further comprises one or more cosmetic, pharmaceutical or other topical agent.

14. The composition of claim 13, wherein the cosmetic, pharmaceutical or other topical agent is selected from the group consisting of agents that improve or eradicate age spots, keratoses and wrinkles; local analgesics and anesthetics; antiacne agents; antibacterials; antiyeast agents; antifungal agents; antiviral agents; antidandruff agents; antidermatitis agents; antihistamine agents; antipruritic agents; antiemetics; antimotionsickness agents; antiinflammatory agents; antihyperkeratolytic agents; antiperspirants; antipsoriatic agents; antiseborrheic agents; hair conditioners and hair treatment agents; antiaging and antiwrinkle agents; sunblock and sunscreen agents; skin lightening agents; depigmenting agents; vitamins; corticosteroids; tanning agents; humectants; hormones; retinoids; gum disease or oral care agents; topical cardiovascular agents; corn, callus and wart removing agents; and dipilating agents.

15. The composition of claim 13, wherein the cosmetic, pharmaceutical or other topical agent is selected from the group consisting of aclovate, acyclovir, acetylsalicylic acid, adapalene, albuterol, aluminum acetate, aluminum chloride, aluminum hydroxide, aluminum chlorohydroxide, amantadine, aminacrine, aminobenzoic acid (PABA), aminocaproic acid, aminosalicylic acid, amitriptyline, anthralin, ascorbic acid, ascoryl palimate, atropine, azelaic acid, bacitracin, bemegride, beclomethasone dipropionate, benzophenone, benzoyl peroxide, betamethasone dipropionate, betamethasone valerate, brompheniramine, bupivacaine, butoconazole, calcipotriene, camphor, capsaicin, carbamide peroxide, chitosan, chlorhexidine, chloroxylenol, chlorpheniramine, ciclopirox, clemastine, clindamycin, clioquinol, clobetasol propionate, clotrimazole, coal tar, cromolyn, crotamiton, cycloserine, dehydroepiandrosterone, desoximetasone, dexamethasone, diphenhydramine, doxypin, doxylamine, dyclonine, econazole, erythromycin, estradiol, ethinyl estradiol, fluocinonide, fluocinolone acetonide, 5-fluorouracil, griseofulvin, guaifenesin, haloprogin, hexylresorcinol, homosalate, hydrocortisone, hydrocortisone 21-acetate, hydrocortisone 17-valerate, hydrocortisone 17-butyrate, hydrogen peroxide, hydroquinone, hydroquinone monoether, hydroxyzine, ibuprofen, ichthammol, imiquimod, indomethacin, ketoconazole, ketoprofen, kojic acid, lidocaine, meclizine, meclocycline, menthol, mepivacaine, methyl nicotinate, methyl salicylate, metronidazole, miconazole, minocycline, minoxidil, monobenzone, mupirocin, naftifine, naproxen, neomycin, nystatin, octyl methoxycinnamate, octyl salicylate, oxybenzone, oxiconazole, oxymetazoline, padimate O, permethrin, pheniramine, phenol, phenylephrine, phenylpropanolamine, piperonyl butoxide, podophyllin, podofilox, povidone iodine, pramoxine, prilocaine, procaine, promethazine propionate, propranolol, pseudoephedrine, pyrethrin, pyrilamine, resorcinol, retinal, 13-cis retinoic acid, retinoic acid, retinol, retinyl acetate, retinyl palmitate, salicylamide, salicylic acid, selenium sulfide, shale tar, sulconazole, sulfur, sulfadiazine, tazarotene, terbinafine, terconazole, tetracaine, tetracycline, tetrahydrozoline, thymol, tioconazole, tolnaftate, triamcinolone diacetate, triamcinolone acetonide, triamcinolone hexacetonide, triclosan, triprolidine, undecylenic acid, urea, vitamin E acetate, wood tar, zinc pyrithione, lactobionic acid, maltobionic acid, glycolic acid, mandelic acid, lactic acid, tropic acid, methyllactic acid, tartaric acid, citric acid, malic acid, glucuronic acid, glucuronolactone, ribonic acid, ribonolactone, gluconic acid, gluconolactone, galactonic acid, galactonolactone, glucarolactone, galactarolactone, N-acetyl-cysteine, N-acetyl-proline, N-acetyl-prolinamide, N-acetyl-lysine, N-acetyl-glutamine, N-acetyl-ornithine and N-acetyl-glucosamine.

16. A method for preventing or treating a cosmetic condition or a dermatological disorder comprising topically administering to a skin area affected with a cosmetic condition or dermatological disorder a composition comprising a therapeutically effective amount of a non-amphoteric glutathione derivative and a topically acceptable vehicle.

17. The method of claim 16, wherein the cosmetic condition or dermatological disorder is selected from signs and changes of skin, nails and hair associated with intrinsic and/or extrinsic aging; inflammation and disturbed keratinization of the skin; defective synthesis of dermal components; dryness or looseness of skin, nail and hair; xerosis; palmar and plantar hyperkeratosis; uneven and rough surface of skin, nail and hair; dandruff; lichen simplex chronicus; keratoses and hyperkeratoses; follicular hyperkeratoses; acne; pseudofolliculitis barbae; dermatoses; eczema; psoriasis; pruritus; warts; herpes; age spots; lentigines; melasmas; blotches; blemished skin; mottled skin; hyperpigmented or darker skin; abnormal or diminished synthesis of collagen, glycosaminoglycans, proteoglycans and elastin; stretch marks; skin lines; fine lines; wrinkles; thinning of skin, nail plate or hair; skin thickening due to elastosis of photoaging; loss or reduction of skin, nail and hair resiliency, elasticity and recoilability; lack of skin, nail and hair lubricants and luster; dull and older-looking skin, nail and hair; fragility and splitting of nail and hair; for skin lightening and wound healing; yellowing skin; reactive, irritating or telangiectatic skin.

18. The method of claim 16, wherein the non-amphoteric glutathione derivative is selected from the group consisting of an N-acyl-glutathione, an N-acyl-glutathione amide, and an N-acyl-glutathione ester.

19. The method of claim 16, wherein the non-amphopteric glutathione derivative is selected from the group consisting of N-acetyl-glutathione; N-acetyl-glutathione monoamide; N-acetyl-glutathione diamide; N-acetyl-glutathione monomethyl ester; N-acetyl-glutathione dimethyl ester; N-acetyl-glutathione monoethyl ester; N-acetyl-glutathione diethyl ester; N-acetyl-glutathione monopropyl ester; N-acetyl-glutathione dipropyl ester; N-acetyl-glutathione monoisopropyl ester; and N-acetyl-glutathione diisopropyl ester.

20. The method of claim 16, wherein the non-amphopteric glutathione derivative is selected from the group consisting of N-propanoyl-glutathione; N-propanoyl-glutathione monoamide; N-propanoyl-glutathione diamide; N-propanoyl-glutathione monomethyl ester; N-propanoyl-glutathione dimethyl ester; N-propanoyl-glutathione monoethyl ester; N-propanoyl-glutathione diethyl ester; N-propanoyl-glutathione monopropyl ester; N-propanoyl-glutathione dipropyl ester; N-propanoyl-glutathione monoisopropyl ester; and N-propanoyl-glutathione diisopropyl ester.

21. The method of claim 16, wherein the non-amphopteric glutathione derivative is selected from the group consisting of N,S-diacetyl-glutathione; N,S-diacetyl-glutathione monoamide; N,S-diacetyl-glutathione diamide; N,S-diacetyl-glutathione monomethyl ester; N,S-diacetyl-glutathione dimethyl ester; N,S-diacetyl-glutathione monoethyl ester; N,S-diacetyl-glutathione diethyl ester; N,S-diacetyl-glutathione monopropyl ester; N,S-diacetyl-glutathione dipropyl ester; N,S-diacetyl-glutathione monoisopropyl ester; and N,S-diacetyl-glutathione diisopropyl ester.

22. The method of claim 16, wherein the non-amphopteric glutathione derivative is selected from the group consisting of N,S-dipropanoyl-glutathione; N,S-dipropanoyl-glutathione monoamide; N,S-dipropanoyl-glutathione diamide; N,S-dipropanoyl-glutathione monomethyl ester; N,S-dipropanoyl-glutathione dimethyl ester; N,S-dipropanoyl-glutathione monoethyl ester; N,S-dipropanoyl-glutathione diethyl ester; N,S-dipropanoyl-glutathione monopropyl ester; N,S-dipropanoyl-glutathione dipropyl ester; N,S-dipropanoyl-glutathione monoisopropyl ester; N,S-dipropanoyl-glutathione diisopropyl ester; glutathione diamide; glutathione dimethyl ester; glutathione diethyl ester; glutathione dipropyl ester; glutathione diisopropyl ester; N-acetyl-S-propanoyl-glutathione; and N-propanoyl-S-acetyl-glutathione.

23. The method of claim 16, wherein the composition further comprises one or more cosmetic, pharmaceutical or other topical agent.

24. The method of claim 23, wherein the cosmetic, pharmaceutical or other topical agent is selected from the group consisting of agents that improve or eradicate age spots, keratoses and wrinkles; local analgesics and anesthetics; antiacne agents; antibacterials; antiyeast agents; antifungal agents; antiviral agents; antidandruff agents; antidermatitis agents; antihistamine agents; antipruritic agents; antiemetics; antimotionsickness agents; antiinflammatory agents; antihyperkeratolytic agents; antiperspirants; antipsoriatic agents; antiseborrheic agents; hair conditioners and hair treatment agents; antiaging and antiwrinkle agents; sunblock and sunscreen agents; skin lightening agents; depigmenting agents; vitamins; corticosteroids; tanning agents; humectants; hormones; retinoids; gum disease or oral care agents; topical cardiovascular agents; corn, callus and wart removing agents; and dipilating agents.

25. The method of claim 23, wherein the cosmetic, pharmaceutical or other topical agent is selected from the group consisting of aclovate, acyclovir, acetylsalicylic acid, adapalene, albuterol, aluminum acetate, aluminum chloride, aluminum hydroxide, aluminum chlorohydroxide, amantadine, aminacrine, aminobenzoic acid (PABA), aminocaproic acid, aminosalicylic acid, amitriptyline, anthralin, ascorbic acid, ascoryl palimate, atropine, azelaic acid, bacitracin, bemegride, beclomethasone dipropionate, benzophenone, benzoyl peroxide, betamethasone dipropionate, betamethasone valerate, brompheniramine, bupivacaine, butoconazole, calcipotriene, camphor, capsaicin, carbamide peroxide, chitosan, chlorhexidine, chloroxylenol, chlorpheniramine, ciclopirox, clemastine, clindamycin, clioquinol, clobetasol propionate, clotrimazole, coal tar, cromolyn, crotamiton, cycloserine, dehydroepiandrosterone, desoximetasone, dexamethasone, diphenhydramine, doxypin, doxylamine, dyclonine, econazole, erythromycin, estradiol, ethinyl estradiol, fluocinonide, fluocinolone acetonide, 5-fluorouracil, griseofulvin, guaifenesin, haloprogin, hexylresorcinol, homosalate, hydrocortisone, hydrocortisone 21-acetate, hydrocortisone 17-valerate, hydrocortisone 17-butyrate, hydrogen peroxide, hydroquinone, hydroquinone monoether, hydroxyzine, ibuprofen, ichthammol, imiquimod, indomethacin, ketoconazole, ketoprofen, kojic acid, lidocaine, meclizine, meclocycline, menthol, mepivacaine, methyl nicotinate, methyl salicylate, metronidazole, miconazole, minocycline, minoxidil, monobenzone, mupirocin, naftifine, naproxen, neomycin, nystatin, octyl methoxycinnamate, octyl salicylate, oxybenzone, oxiconazole, oxymetazoline, padimate O, permethrin, pheniramine, phenol, phenylephrine, phenylpropanolamine, piperonyl butoxide, podophyllin, podofilox, povidone iodine, pramoxine, prilocaine, procaine, promethazine propionate, propranolol, pseudoephedrine, pyrethrin, pyrilamine, resorcinol, retinal, 13-cis retinoic acid, retinoic acid, retinol, retinyl acetate, retinyl palmitate, salicylamide, salicylic acid, selenium sulfide, shale tar, sulconazole, sulfur, sulfadiazine, tazarotene, terbinafine, terconazole, tetracaine, tetracycline, tetrahydrozoline, thymol, tioconazole, tolnaftate, triamcinolone diacetate, triamcinolone acetonide, triamcinolone hexacetonide, triclosan, triprolidine, undecylenic acid, urea, vitamin E acetate, wood tar, zinc pyrithione, lactobionic acid, maltobionic acid, glycolic acid, mandelic acid, lactic acid, tropic acid, methyllactic acid, tartaric acid, citric acid, malic acid, glucuronic acid, glucuronolactone, ribonic acid, ribonolactone, gluconic acid, gluconolactone, galactonic acid, galactonolactone, glucarolactone, galactarolactone, N-acetyl-cysteine, N-acetyl-proline, N-acetyl-prolinamide, N-acetyl-lysine, N-acetyl-glutamine, N-acetyl-ornithine and N-acetyl-glucosamine.
